(19) United States
(12) Patent Application Publication
(10) Pub. No.: US 2007/0161924 A1
(43) Pub. Date: Jul. 12, 2007

(54) Ear probe and disposable ear tip system

(76) Inventors: William F. Dolphin, Dalkeith (AU); Andrew Quick, Sudbury, MA (US)

Correspondence Address: LUCASH, GESMER & UPDEGROVE, LLP, 40 BROAD ST, SUITE 300, BOSTON, MA, 02109, US

(21) Appl. No.: 10/557,743

(22) PCT Filed: May 21, 2004
(86) PCT No.: PCT/US04/16200

§ 371 Date: Nov. 21, 2005

Related U.S. Application Data
(60) Provisional application No. 60/473,297, filed on May 23, 2003.

(51) Int. Cl. A61B 5/00

(52) U.S. Cl. 600/559

(57) ABSTRACT

An improved ear probe and disposable ear tip system for insertion into an ear canal of a subject during auditory testing. The probe includes a base portion and a cap portion. The base portion of the probe houses electronic components for sound generation, transmission and collection. The cap portion of the probe includes features for mechanical attachment of the tip to the probe in such a way as to provide a tight acoustic seal between the probe and the tip. The tip is disposable and protects the probe from being occluded by debris in the ear canal.

### RELATED APPLICATIONS

This application claims priority to U.S. provisional application Ser. No. 60/473,297, filed on May 23, 2003 and entitled “An Improved Ear Probe and Disposable Ear Tip System.”

### FIELD OF THE INVENTION

The invention generally relates to the field of ear probes and disposable ear tips used in acoustical and visual examinations of the ear and related physiology. In particular, the invention relates to an improved ear probe and tip system that maintains a tension fit between the probe and the disposable tip.

### BACKGROUND OF THE INVENTION

Ear probe systems that include an ear probe and a disposable tip are known in the art. Such systems are typically used in combination with devices that test for hearing loss and/or ear pathologies (e.g., audiometric screening apparatus). For example, ear probe systems can be used to test a subject's hearing function, such as a test of a subject's otoacoustic emission (OAE), a test of a subject's auditory evoke potentials (AEP), a test of a subject's acoustic reflectivity (AR), a test of a subject's otoacoustic reflectance (OR), and/or a test of a subject's tympanometry. These hearing tests involve delivery of a controlled acoustic stimulus through the ear probe system and/or acoustic measurements made by collecting sound within the subject's ear canal. The ear probe of the system serves as a reusable device for delivering and collecting data to and from a subject's ear. The disposable ear tip serves as a conduit of acoustic signals into and out of the subject's ear. In addition to transmitting and receiving acoustic signals, the disposable ear tip serves to seal off the subject's ear canal from ambient noises, which could affect the results of the hearing test being performed. Disposable ear tips have also been used to prevent ear canal collapse in infants.

A number of disadvantages and limitations exist with respect to known ear probe systems. For example, in some ear probe systems the ear tips form a sleeve over the probe extension. The ear tip receives the probe, whereby the ear tip can be positioned fully over the probe or probe extension while still allowing the ear tip's end to slightly extend past the end of the probe. If the probe extension does not run the entire length of the ear tip, then a cavity is formed at the distal end (extending from the end of the ear tip to the end of the probe) in which outgoing and incoming acoustic signals mix giving rise to artifacts, namely acoustic beats and other nonlinear distortion products. Theses artifacts can be picked up by the probe's microphone and can interfere with data collection, which affects the test results. In other ear probe systems, the probe includes channels that extend the entire length of an attached ear tip. As a result, the channels in the probe are exposed to the subject's ear canal including debris (e.g., vernix and cerumen) and/or fluid residing therein. Not only does this exposure contaminate the probe, but also this exposure can lead to occlusion and damage of the probe. Moreover, a probe including one or more channels extending through the entire length of the tip presents a potential risk of damage to the tissue of the inner walls of the subject's ear canal because the material forming the probe is typically less compliant than the material forming the disposable tip. As a result, such systems are likely to scrape or bruise tissue lining the subject's ear if inserted incorrectly (e.g., at an improper angle and/or with excessive force).

Another problem associated with conventional ear probe systems is misalignment of the ear probe and ear tip. In some systems, ear tips are manually fitted over probe speakers and microphone channels. Such configurations require a user to carefully align features (e.g., channels) defined within the tip with corresponding features (e.g., mating features) extending from the probe. Even with careful application of the tip to the probe, the channels within the probe often scrape or core the material forming the tip. As a result, the material scraped or cored from the tip can become lodged within the probe, damaging and/or rendering the ear probe system inoperable.

Some previous ear probe systems also suffer from an inability to adapt to various sized ear canals. As a result, the user conducting the hearing test must select from among a multitude of ear tips to select the correct sized ear tip prior to positioning the ear tip on the probe and conducting the examination. Selection of an appropriately sized ear tip requires careful visual inspection of the ear canal and considerable clinical experience and acumen. If an incorrectly sized ear tip is selected, the tip will fail to provide an adequate acoustic seal between the ear canal and the external environment. In addition, an incorrectly sized ear tip may work loose and fall out of the ear canal during an examination.

### SUMMARY OF THE INVENTION

In general, in one aspect, the present invention features an improved ear probe. The ear probe includes a base portion and a cap portion. The base portion houses at least one signal transducing device, such as, for example, a microphone or a speaker. The cap portion extends from the base portion and defines one or more channels for signal transmission and a plurality of pedestal surfaces. As used herein, a pedestal surface is the surface positioned above an underlying pedestal.

Embodiments of this aspect of the invention can include one or more of the following features. The cap portion can include a protrusion extending away from the cap portion at an angle (e.g., 30°, 45°, 60°, 80°, 90°) to a longitudinal axis of the channel of the cap portion. The protrusion can be angled and in some embodiment, the protrusion can encircle the cap portion. The probe can also include an alignment feature that inhibits radial rotation of an ear probe tip attached to the ear probe about a longitudinal axis of the channel defined in the cap portion. In certain embodiments, the alignment feature is a fin. In some embodiments, the aligmnent feature is a projection extending from a proximal end of the cap portion. The probe can also include a light source, such as, for example, a light emitting diode, to aid in a visual inspection of a subject's ear.

In another aspect, the invention features an ear probe including a base portion and a cap portion. The base portion houses at least one signal transducing device. The cap portion extends away from the base portion and includes at least one pedestal portion including a protrusion extending away from the pedestal portion at an angle to a longitudinal axis of the cap portion.

Embodiments of this aspect of the invention can include one or more of the following features. The ear probe can include a plurality of protrusions extending from the pedestal portion. The one or more protrusions can be angled. In some embodiments, the protrusion is a ring encircling the pedestal portion. In certain embodiments, the cap portion of the probe can define at least two channels for signal transmission. The cap portion can further include an alignment feature that inhibits radial rotation of an ear probe tip attached to the ear probe about the longitudinal axis of the cap portion. Examples of alignment features include a fin positioned between the two channels and a projection extending from a proximal end of the cap portion. The probe can further include a light source, such as, for example, a light emitting diode, to aid in a visual inspection of a subject's ear.

In another aspect, the invention features an ear probe including a base portion and a cap portion. The base portion houses at least one signal tranducing device (e.g., a microphone or a speaker). The cap portion defines at least two channels for signal transmission and includes an alignment feature that inhibits radial rotation of an ear probe tip secured to the cap portion about an axis extending between a distal end of the cap portion and a proximal end of the cap portion. In certain embodiments, the alignment feature is a fin disposed between the two channels defined in the cap portion. In some embodiments, the alignment feature is a projection extending from the proximal end of the cap portion.

Embodiments of this aspect of the invention can include one or more of the following features. The cap portion can further include one or more pedestal portions. The pedestal portion can include a protrusion extending away from the pedestal portion at an angle to the axis extending between the distal end of the cap portion and the proximal end of the cap portion. The probe can further include a light source, such as, for example, a light emitting diode, to aid in a visual inspection of a subject's ear.

In another aspect, the invention features an ear probe including a base portion and a cap portion extending from the base portion. The base portion includes means for transmitting signals to the ear and the cap portion includes a first means for securing the tip to the cap portion and a second means for preventing radial rotation of the secured tip about the cap portion.

In general, in another aspect, the invention features a tip for an ear probe. The tip includes a distal end, a proximal end, and a body extending between the distal and proximal ends. The body includes an exterior surface and an interior surface. The interior surface of the body defines at least two channels and an alignment slot for providing proper positioning of the ear probe tip to the ear probe.

Embodiments of this aspect of the invention can include one or more of the following features. One or more portions of the exterior surface of the tip can include a texture, such as a random or a repeating pattern of elements disposed about the distal tip. In certain embodiments, the texture is formed of microbumps. The microbumps may be continuous about the circumference of the tip. The tip can be disposable. In some embodiments, the tip can include a feature, such as an aperture, that mechanically weakens the tip so that the tip tears upon removing the tip from the ear probe.

In another aspect, the invention features a tip for an ear probe. The tip includes a distal end, a proximal end, and a body extending between the distal and proximal ends. The body includes a means for coupling with the ear probe so that proper alignment between the tip and the probe is ensured.

In general, in another aspect, the invention features an ear probe system. The ear probe system can be used in devices that are inserted into a subject's ear, such as, for example, audiometric screening devices, headphones for stereo equipment, cell phones, and hearing aids. The ear probe system includes a probe and a tip, which can be disposable. The probe includes a base portion that houses at least one signal transducing device (e.g., a microphone or a speaker) and a cap portion that extends from the base portion. The cap portion defines a first channel and includes a protrusion extending away from the cap portion at an angle to a longitudinal axis of the channel. The tip of the system defines a second channel. The tip is secured to the cap portion of the probe so that the protrusion maintains a tension fit between the probe and the tip to create a continuous channel extending from the first channel to the second channel.

Embodiments of this aspect of the invention can include one or more of the following features. In some embodiments, no portion of the first channel extends into the second channel when the tip is secured to the probe. In some embodiments, no portion of the second channel extends into the first channel when the tip is secured to the probe. In certain embodiments, the second channel (the channel defined in the tip) is sized to prevent the first channel (the channel defined in the probe) from contacting debris in an ear of a subject when the tip is secured to the probe and positioned in the subject's ear. In certain embodiments, the protrusion is angled. In some embodiments, the protrusion is a ring encircling the pedestal portion. The system can include a visual guide feature to provide proper radial alignment of the tip to the probe. In some embodiments, the visual guide feature includes a projection extending from the probe and a corresponding cutout disposed on the tip. The system can further include a light source, such as, for example, a light emitting diode, to aid in a visual inspection of a subject's ear.

In another aspect, the invention features an ear probe system including a probe and a tip. The probe defines a first probe channel and a second probe channel and includes a first alignment feature. The tip defines a first tip channel and a second tip channel and includes a second alignment feature. When the tip is secured to the probe, the first alignment feature of the probe mates with the second alignment feature of the tip to ensure alignment of the first probe channel with the first tip channel.

In another aspect, the invention features an ear probe system. The ear probe system includes a probe defining a first probe channel and a second probe channel, a tip defining a first tip channel and a second tip channel, and means for automatic alignment of the first probe channel to the first tip channel when the tip is positioned on the probe.

In another aspect, the invention features an ear probe system including a probe and a tip. The probe includes a base portion housing at least one transducing device and a cap portion extending from the base portion. The cap portion defines a first probe channel and a second probe channel and includes a protrusion and a tip alignment feature that is disposed between the first and second probe channels. The tip of the system includes an exterior surface and an interior surface. The interior surface defines a first tip channel, a second tip channel and a probe alignment slot. The tip alignment feature has a greater length along a longitudinal direction of the ear probe system than the probe alignment slot so as to stretch the tip over the protrusion during attachment of the tip to the cap portion of the probe.

In general, in yet another aspect the invention relates to a method of attaching a tip to a probe. The method includes the following steps. A probe is provided. The probe includes a base portion housing at least one signal transducing device and a cap portion extending from the base portion. The cap portion defines a first probe channel and a second probe channel and includes a protrusion and a tip alignment feature which is disposed between the first and second probe channels. A tip is positioned on the probe. The tip includes an exterior surface and an interior surface. The interior surface of the tip defines a first tip channel, a second tip channel, and a probe alignment slot. The probe alignment slot has a smaller length along a longitudinal direction of the ear probe system than the tip alignment feature of the probe. The tip is stretched over the tip alignment feature of the probe such that a portion of the tip is secured to the probe by the protrusion.

In general, the probe and tip forming the system can include one or more of the following advantages. The ear probe system can be used to perform auditory and/or visual examinations of ears of various sizes. For example, the ear probe system including texturing on a tip's external surface can be used to test the hearing function of an infant's ear, a child's ear, a young adult's ear and/or an adult's without any modification to the probe and/or the tip. Another advantage of the probe and tip system is probe cleanliness. The ear probe system's tip covers and protects the probe from coming into direct contact with the subject's ear canal. As a result, only the tip, which is disposable, is contaminated by debris residing in the subject's ear.

A further advantage of the ear probe and tip system is user ease. The tip and probe can include features that ensure proper alignment of the tip to the probe. As a result, the user can easily attach the tip and probe together without having to spend a considerable amount of time and effort trying to properly align features in the tip with the probe. In addition, the probe can include a protrusion or other pressure-fit locking mechanisms that maintain a tension fit between the probe and the ear tip. As a result of this improved attachment and locking mechanism, the system maintains acoustic isolation between channels (e.g., no cavities are formed between a channel in the probe and a corresponding channel defined in the tip). As a further result, shearing or coring of the tip is avoided during positioning and attachment of the tip to the probe because the probe channels are not inserted into channels defined within the tip, but rather the channels in the probe and the corresponding channels in the tip are brought and held in contact by the tension fit.

Another advantage of the ear probe and tip system is mechanical stability. In some embodiments, the probe, which is typically formed from a rigid. plastic, includes one or more pedestal portions that extend into the disposable tip's interior. As a result, the pedestal portions reinforce the disposable tip, thereby preventing the tip from collapsing or kinking.

The foregoing and other aspects, features, and advantages of the invention will become more apparent from the following description and from the claims.

### BRIEF DESCRIPTION OF THE DRAWINGS

The foregoing and other features and advantages of the invention, as well as the invention itself will be more fully understood from the following illustrative description, when read together with the accompanying drawings which are not necessarily to scale.

FIG. 1 is an illustration of an audiometric screening apparatus including a ear probe system in accordance with an embodiment of the invention being inserted into a test subject's ear.

FIG. 2 is an illustration of the portable audiometric screening apparatus of FIG. 1.

FIG. 3 is a cross-sectional view of an ear probe system, including an ear probe and a disposable tip, according to an illustrative embodiment of the invention.

FIG. 4 is a perspective view of an ear probe of an ear probe system, according to an illustrative embodiment of the invention.

FIG. 5 is a perspective view of a disposable tip for an ear probe system, according to an illustrative embodiment of the invention.

### DETAILED DESCRIPTION OF ILLUSTRATIVE EMBODIMENTS

In some embodiments, the invention relates to an ear probe system used in combination with an audiometric screening apparatus, such as, for example, the apparatuses described in U.S. Pat. Nos. 5,601,091 and 5,916,174 and herein incorporated by reference in their entirety. The ear probe system includes a probe and a disposable tip. The probe includes a base portion housing at least one signal transducing device (e.g., a transducer, such as a microphone or a speaker) and a cap portion defining a channel in which signals can be transmitted to the ear and/or received from the ear. The disposable tip is attached to the cap portion of the probe. During an auditory examination, the tip protects the probe from contamination and damage while allowing signals to pass without interference between the probe and the ear.

The ear probe system of the invention can be used to perform a variety of tests including, but not limited to, OAE testing, AEP testing, AR testing, OR testing, and tympanometry testing. OAE testing takes advantage of nonlinearities in a healthy auditory system in obtaining OAE signals. The procedure requires that an acoustic stimulus signal be presented to the subject's ear. The acoustic energy is conducted, via structures of the middle ear, to the fluid filled cochlea. Pressure waves propagating within the fluid result in displacements of the basilar membrane. Such displacements cause excitation of the inner and outer hair cells. It is believed that, due to active processes primarily associated with length changes in outer hair cells, energy is retransmitted in retrograde manner out of the cochlea and conducted through the middle ear to the eardrum which acts as a speaker, producing acoustic energy detectable in the ear canal. Due to nonlinearities of the ear, this retransmitted sound, which is measured as an OAE, occurs at frequencies other than those present in the original stimulus.

The AEP test is a noninvasive procedure in which an acoustic stimulus signal, such as a brief click or toneburst, is presented to the subject's ear to elicit an auditory evoked potential. Auditory evoked potentials are electrical responses of cells within the subject's auditory pathway of the brain to an acoustic stimulus. These electrical responses are recorded from the scalp using electrodes. In a normally functioning auditory system, a suprathreshold sound stimulates cells within the auditory pathway of the brain (primarily neurons comprising the auditory nerve and brainstem structures). This excitation spreads from the peripheral to more central structures resulting in the discharge of large numbers of neurons within the pathway. The neural activity is time-locked to the acoustic stimulus signal resulting in the synchronized discharge of large neuronal assemblies. As excitation moves through the auditory system, a sequential pattern of electrical potentials are measured from the scalp which appear as a highly stereotypical series of waves on an electroencephalogram. Analysis of these acoustically evoked brainwave patterns can be used to determine if the auditory system is functioning normally.

AR tests measure the degree to which sound across a range of frequencies is reflected off of the tympanum. A stimulus signal is transmitted into the subject's ear, and a AR response signal is received therefrom. The AR signal provides an indication of the presence of fluid in the middle ear cavity.

Tympanometry tests are used to measure the acoustic admittance (or absorption) of the tympanic membrane and middle ear system at select frequencies over a range of atmospheric pressures. Tympanometry devices typically serve as diagnostic instruments for detecting the presence of fluid in the middle ear cavity.

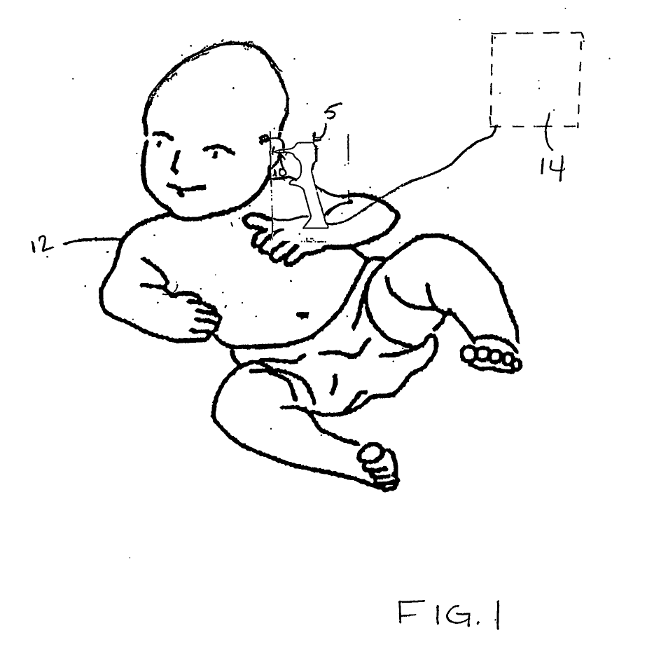

FIGS. 1 and 2 illustrate an audiometric screening apparatus 5 incorporating an ear probe and tip system 10. As shown in FIG. 1, the ear probe and tip system 10 of the apparatus 5 is inserted into an ear of a subject 12 to test hearing function. In some embodiments, data collected by the apparatus 5 can be transmitted to a docking station 14 through a wire or wirelessly. Referring in particular to FIG. 2, the ear probe and tip system 10 includes a reusable ear probe 15 for transmitting and receiving signals to and from the subject's ear and a disposable tip 20 attached to the probe 15. The apparatus 5 further includes a hand held housing 22 and a docking station 14 (FIG. 1) for receiving the housing. Disposed within the housing 22 is LCD screen 24 for displaying test results, a signal processor for generating signals, batteries, recharging circuitry, data transfer circuitry, a pressure pump and air cavities. Push button controls 26 are conveniently located on the housing 22 for operation of apparatus 5. The tip and probe system can be attached to the body of the apparatus as shown. Alternatively, the tip and probe system can be attached via a flexible cable connector.

In one configuration, the docking station 14 includes a battery charger and a printer. Data collected from the probe 15 is simply passed to the printer. In another configuration, the docking station includes the battery charger, printer, a signal processor to analyze signals, storage devices (e.g., hard disk and disk drive), and serial and/or parallel ports for communication with other processors. Communication with printers, storage devices, or any other element of the docking station can be via any form of wired or wireless transmission.

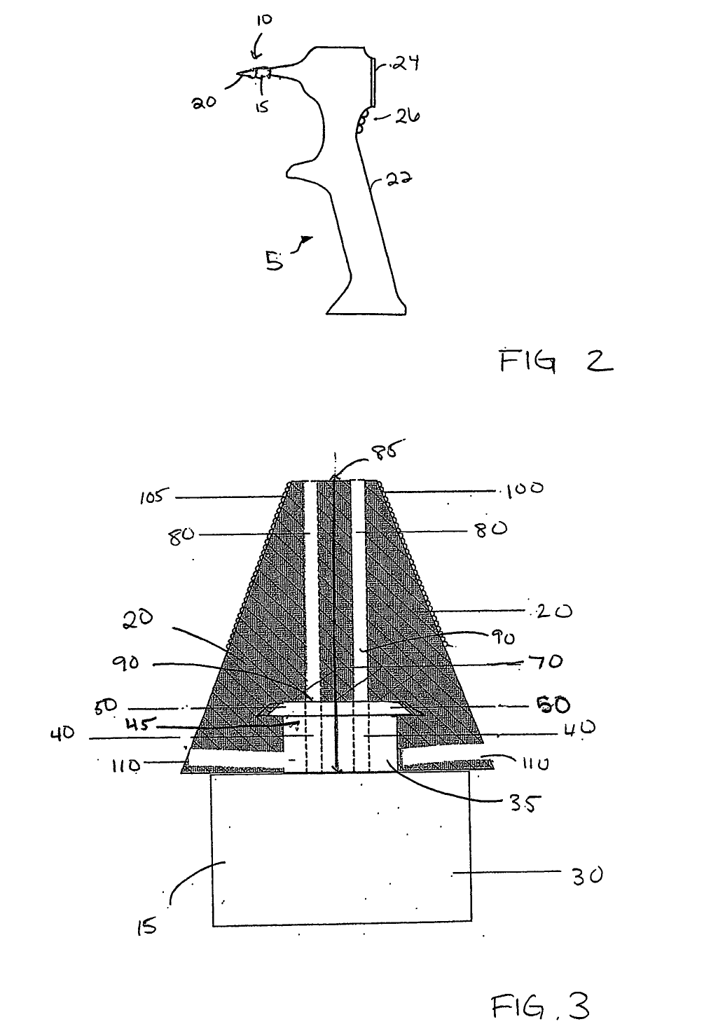

The probe and tip system 10, which is shown in cross-section and removed from the apparatus 5 in FIG. 3, is inserted into a subject's ear to perform an appropriate hearing test (e.g., OAE, ABR, AR, tymanometry). The probe 15 includes a base portion 30 that surrounds one or more signal transducing devices (e.g., microphones, speakers). The probe 15 also includes a cap portion 35 extending from the base portion 30. The cap portion 35 defines signal transmitting channels 40 (e.g., here, two) that extend from the devices in the base 30 through the cap portion 35. In addition to the channels 40, the cap portion 35 includes a pedestal portion 45 having an angled rim 50 (e.g., a protrusion or an undercut) that extends away from the pedestal portion at an angle of about 90°. The disposable tip 20 fits over the pedestal portion 45 and is secured to the probe 15 by the angled rim 50. The securing force of the angled rim 50 provides a downward tension on the disposable tip 20, thereby locking the tip 20 to the cap portion 35 along pedestal surface 70 to ensure a complete acoustic seal between the tip 20 and the probe 15. The disposable tip 20 includes tip channels 80 that extend along a longitudinal length 85 of the tip 20. When the tip 30 is secured to the probe 15, the tip channels 80 align with the channels 40 defined in the cap portion 35 to form continuous discrete passages 90 therethrough. The complete acoustic seal formed along pedestal surface 70 prevents leakage of acoustical energy between the passages 90 and thus, ensures that the passages 90 are acoustically isolated from each other.

The attached disposable tip 20 is typically formed of a compliant, biocompatible material, such as, for example a polyolefin polymer (e.g., polyethylene), silicone polymer, or any other flexible, low durometer, biologically inert material. The tip 20 is designed to be soft and flexible so as not to injure the subject's ear. As a result, a detached tip 20 can be prone to bending and kinking. To support and reinforce the tip so as to prevent the tip from bending during an auditory examination, the pedestal portion 45 of the probe extends into the tip 20. The pedestal portion 45, as well as the cap portion 35 and the base portion 30 of the probe 15 are formed of a rigid material, such as, for example plastic or metal. Thus, the pedestal portion 45 residing inside of the proximal end of the tip 20 reinforces the tip to such an extent that bending and kinking of the tip is potentially eliminated.

The disposable tip 20 may further include texturing 100 on its external surface. In the embodiment shown in FIG. 3, the disposable tip 20 has a texture that includes a plurality of discrete microbumps 105 covering the distal end of the tip 20. In other embodiments, the texturing can be provided along the entire length of the tip 20. In certain embodiments, the texture can have a predetermined pattern (e.g., the microbumps form a repeating pattern). In other embodiments, the texture has a random or non-repeating pattern. In some embodiments, the texturing can be continuous about the circumference of the tip. For example, the texture can include a plurality of microgroves (e.g., groves cut into a portion of the tip) or a plurality of microridges (e.g., ridges of material extending from a portion of the tip) that encircle the distal end of the tip. As used herein, the modifier “micro,” such as microridges and microgroves defines features that have a size defined in microns (e.g., 0.1 micron to 5000 microns, 1 micron to 1000 microns).

The texture on the external surface of the tip 20 can serve several functions. First, the texture can provide the tip 20 with tacky surface that secures the tip within the subject's ear canal and prevents the ear probe and tip system 10 from becoming dislodged during an examination. In addition, the textured surface allows the tip 20 to be used in subjects of various sizes without compromising fit. For example, tip 20 including microbumps can be used in both infant and adult subjects. Finally, the textured surface occludes the ear canal to eliminate ambient noise within the ear canal.

The disposable tip 20 may further include apertures 110. Apertures 110 are designed to mechanically weaken the disposable tip so that the tip 20 tears upon removal from the probe 15, thereby ensuring that used tips are not recycled.

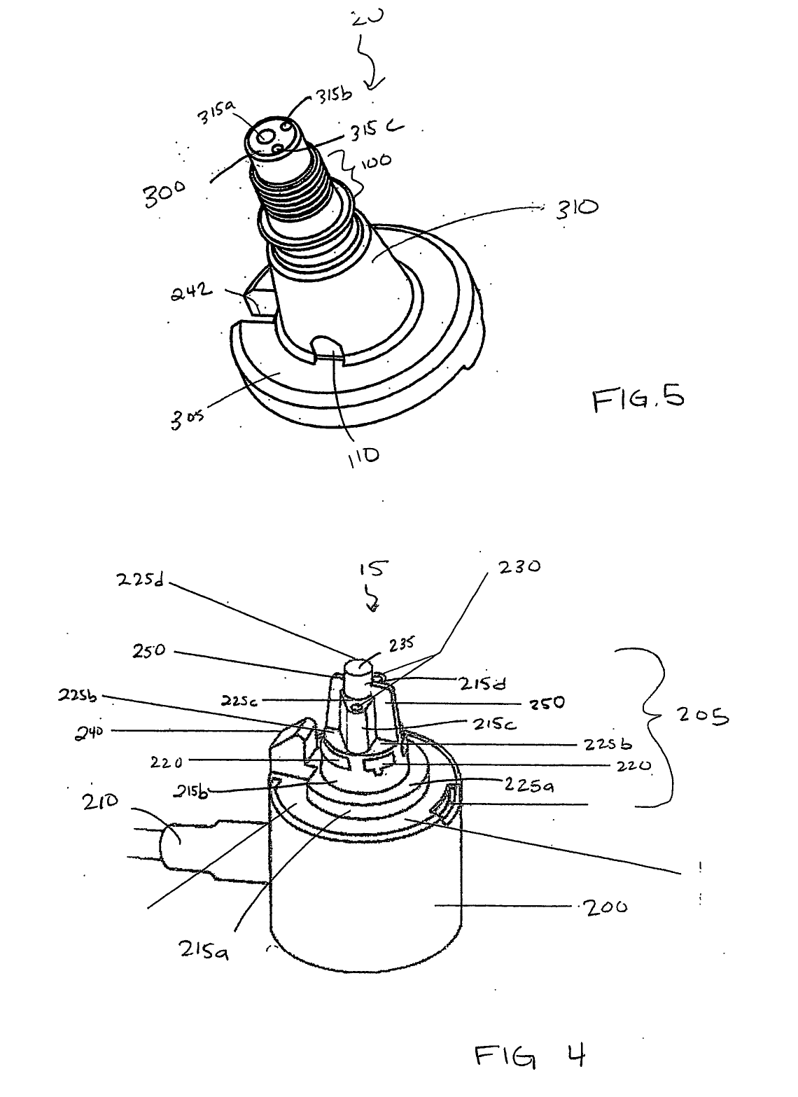

Referring to FIGS. 4 and 5, other embodiments of the probe 15 and tip 20 forming system 10 are shown. In the embodiment shown in FIG. 4, probe 15 includes a base portion 200 and a cap portion 205. The base portion includes signal transducing elements which are attached to a signal processor via cable 210. Cap portion 205 extends from base portion 200 and includes four pedestal portions 215a, b, c, d. About the base of one of the pedestal portions, 215b, multiple protrusions 220 project out from the probe at angle to secure a disposable tip, such as the tip shown in FIG. 5, to the probe 15. The pedestal portions 215a, b, c, and d each define a pedestal surface 225a, b, c, and d that are located at a different heights along the longitudinal length of the probe 15. Each pedestal surface 225a, b, c, and d can provide a different function to the probe 15. For example, pedestal surface 225b provides an area in which an acoustical seal is formed (e.g., tension fit is formed), pedestal surface 225c is provided with two speaker output ports 230, and pedestal surface 225d is provided with an input port 235. As a result of this configuration, the interface between the channels in the tip 20 and the probe 15 for the signals received and the signals transmitted are on different levels, which helps to maintain acoustical isolation within the system 10.

Because proper alignment of the tip 20 and the probe 15 is important for obtaining accurate hearing test results, the probe 15 can include several features to aid a user during alignment. One such feature includes a projection 240 extending from the proximal end of the cap portion 205. The projection 240 mates with a corresponding cut out 242 in the disposable tip (see FIG. 5) and provides a visual and tactile guide to the user during alignment. In addition to providing assistance to the user during alignment, once the tip is secured to the probe, the projection 240 inhibits radial rotation of the tip 20 about the probe 15. Another alignment feature which may be included in the probe 15 is a fin 250. Fin 250 is positioned between two signal transmitting channels in communication with speaker output ports 230. The fin 250 has a cross-sectional area that is defined by a short axis and a long axis. The disposable tip 20 (FIG. 5) used with the probe shown in FIG. 4 includes a probe alignment slot (not shown) defined within its interior that has the same cross-sectional shape as fin 250. When the tip is first inserted on to the probe, the tip will be able to rotate about the probe 15 until the fin 250 aligns with the probe alignment slot. Once the fin 250 and the probe alignment slot are aligned, the user can pull the tip 20 over the cap portion 205 of the probe 15 to create an acoustical seal therebetween and to form continuous discrete passages extending through the tip 20 and the cap portion 205. During attachment, fin 250 is inserted into the probe alignment slot of the tip 20 so as to lock the radial position of the tip with respect to the probe. As a result, the attached tip is unable to rotate about the probe.

Referring to FIG. 5, the disposable tip 20 used in combination with the probe 15 shown in FIG. 4 includes a distal end 300, a proximal end 305, and a body 310 defining three signal transmitting channels 315a, b, and c. When attached to probe 15, signal transmitting channel 315 a aligns with input port 235, and signal transmitting channels 315b and c align with corresponding speaker output ports 230 to form a continuous passageway from the distal end 300 through cap portion 205 (see FIG. 4) to the transducers located within base portion 200 of probe 15.

Tip 20 can also include aperture 110 that mechanically weakens the tip to such an extent that the tip 20 tears when removed from an attached probe 15 and texturing 100 on its external surface.

In general, the tip and the probe can be formed by using standard manufacturing techniques such as, for example, molding and extrusion. For example, to form a disposable tip including features defined in its external and internal surfaces, shapeable material can be inserted into a mold that is patterned with the features of the external surface of the tip, while a mold insert patterned with the internal features of the tip can be inserted into the center of the mold.

The present invention also features a method of attaching a disposable tip to an ear probe. In one embodiment, the method includes the following steps. First, a user obtains a probe, such as the probe shown in FIG. 4, which includes protrusions 220 and fin 250. Next, the user inserts a disposable tip onto the distal end of the probe. The disposable tip has an interior surface that defines a probe alignment slot, which corresponds to the cross-sectional shape as fin 250 to provide the ear probe and tip system with proper radial alignment The probe alignment slot defined within the tip, while having a substantially similar cross-sectional shape to fin 250, has a smaller longitudinal length than the fin 250. As a result, as the user pulls the tip over the fin 250, the disposable tip stretches to extend along the entire length of fin 250 and protrusions 220. As a further result, once the tip is secured to the probe via the protrusions 220, a tension-fit is created by the force expended to stretch the tip over the fin 250.

While certain embodiments have been described, other embodiments are also possible. As an example, while a probe and tip system has been described as being using to deliver and receive auditory signals, the probe and tip system can be used to deliver other types of energy signals. For example, the probe and tip system can be modified to include a light source, such as a high intensity light emitting diode, and a passageway therethrough to deliver light to the ear for a visual examination of the ear.

As an additional example, while the probe and tip system has been described as being used in an audiometric screening apparatus, the probe and tip system can be used in other devices inserted into an ear, such as, for example, within headphones for stereo equipment, cell phones, or hearing aids.

As a further example, while in some embodiments the probe defines more than one channel for transmitting signals to the ear, in other embodiments, the probe has a single channel for transmitting signals to the ear.

As an additional example, while in some embodiments, an alignment feature included on the probe was defined as a fin having a cross-sectional shape having a long axis and a short axis, other alignment features can be used in addition to or in replacement of the fin. For example, in certain embodiments, the probe can include an alignment feature which has a half-moon cross-sectional shape.

Variations, modifications, and other implementations of what is described herein will occur to those of ordinary skill without departing from the spirit and the scope of the invention. Accordingly, the invention is not to be defined only by the preceding illustrative description.

What is claimed is:

1. An ear probe comprising:
a base portion housing at least one signal transducing device; and
a cap portion extending from the base portion, the cap portion defining a channel for signal transmission and a plurality of pedestal surfaces.

2. The ear probe of claim 1, wherein the cap portion includes a protrusion extending away from the cap portion at an angle to a longitudinal axis of the channel of the cap portion.

3. The ear probe of claim 2, wherein the angle is greater than 45 degrees.

4. The ear probe of claim 2, wherein the protrusion is angled.

5. The ear probe of claim 1, wherein the cap portion includes an alignment feature that inhibits radial rotation of an ear probe tip attached to the ear probe about a longitudinal axis of the channel.

6. The ear probe of claim 5, wherein the alignment feature is a fin.

7. The ear probe of claim 5, wherein the alignment feature is a projection extending from a proximal end of the cap portion.

8. The ear probe of claim 1, wherein the signal transducing device is a microphone.

9. The ear probe of claim 1, wherein the signal transducing device is a speaker.

10. The ear probe of claim 1, further comprising a light source to aid in a visual inspection of an ear of a subject.

11. The ear probe of claim 10, wherein the light source is a light emitting diode.

12. An ear probe comprising:
a base portion housing at least one signal transducing device; and
a cap portion extending from the base portion, the cap portion comprising at least one pedestal portion including a protrusion extending away from the pedestal portion at an angle to a longitudinal axis of the cap portion.

13. The ear probe of claim 12, wherein the at least one pedestal portion includes a plurality of protrusions.

14. The ear probe of the claim 12, wherein the protrusion is angled.

15. The ear probe of claim 14, wherein the protrusion is a ring encircling the pedestal portion.

16. The ear probe of claim 12, wherein the cap portion defines at least two channels for signal transmission.

17. The ear probe of claim 16, wherein the cap portion includes an alignment feature that inhibits radial rotation of an ear probe tip attached to the ear probe about the longitudinal axis of the cap portion.

18. The ear probe of claim 17, wherein the alignment feature is a fin.

19. The ear probe of claim 18, wherein the fin is positioned between the at least two channels.

20. The ear probe of claim 17, wherein the alignment feature is a projection extending from a proximal end of the cap portion.

21. The ear probe of claim 12, wherein the cap portion includes a plurality of pedestal portions.

22. The ear probe of claim 12, wherein the signal transducing device is a microphone.

23. The ear probe of claim 12, wherein the signal transducing device is a speaker.

24. The ear probe of claim 12, wherein the signal transducing device is a light source.

25. The ear probe of claim 24, further comprising a light source to aid in a visual inspection of an ear of a subject.

26. An ear probe comprising:
a base portion housing at least one signal transducing device; and
a cap portion defining at least two channels for signal transmission, the cap portion including an alignment feature that inhibits radial rotation of an ear probe tip secured to the cap portion about an axis extending between a distal end of the cap portion and a proximal end of the cap portion.

27. The ear probe of claim 26, wherein the alignment feature is a fin.

28. The ear probe of claim 27, wherein the fin is disposed between the at least two channels.

29. The ear probe of claim 26, wherein the alignment feature is a projection extending from the proximal end of the cap portion.

30. The ear probe of claim 26, wherein the cap portion includes a pedestal portion.

31. The ear probe of claim 30, wherein the cap portion includes a protrusion extending away from the pedestal portion at an angle to the axis.

32. The ear probe of claim 26, wherein the cap portion includes a plurality of pedestal portions.

33. The ear probe of claim 26, wherein the signal transducing device is a microphone.

34. The ear probe of claim 26, wherein the signal transducing device is a speaker.

35. The ear probe of claim 26, further comprising a light source to aid in a visual inspection of the an ear of a subject.

36. The ear probe of claim 35, wherein the light source is a light emitting diode.

37. An ear probe comprising:
a base portion including means for transmitting signals to the ear; and
a cap portion extending from the base portion, the cap portion including a first means for securing a tip to the cap portion and a second means for preventing radial rotation of the secured tip about the cap portion.

38. A tip for an ear probe, the tip comprising:
a distal end;
a proximal end; and
a body extending between the distal end and the proximal end, the body including an exterior surface and an interior surface, the interior surface defining at least two channels and an alignment slot for providing proper positioning of the ear probe tip to the ear probe.

39. The tip of claim 38, wherein at least a portion of the exterior surface of the body includes a texture.

40. The tip of claim 39, wherein the texture includes microbumps.

41. The tip of claim 40, wherein the microbumps are continuous about the circumference of the tip.

42. The tip of claim 38, wherein the tip is disposable.

43. The tip of claim 42, wherein the body further includes a feature that mechanically weakens the tip so that the tip tears upon removing the tip from the ear probe.

44. The tip of claim 43, wherein the feature is an aperture.

45. A tip for an ear probe, the tip comprising:
a distal end;
a proximal end; and
a body extending between the distal end and the proximal end, the body including a means for coupling with the ear probe so that proper alignment between the tip and the ear probe is ensured.

46. An ear probe system comprising:
a probe comprising:
a base portion housing at least one signal transducing device; and
a cap portion extending from the base portion, the cap portion defining a first channel and including a protrusion extending away from the cap portion at an angle to a longitudinal axis of the channel; and
a tip defining a second channel, the tip being secured to the cap portion of the probe such that the protrusion maintains a tension fit between the probe and the tip to create a continuous channel extending from the first channel to the second channel.

47. The ear probe system of claim 46, wherein no portion of the first channel is disposed within the second channel when the tip is secured to the probe.

48. The ear probe system of claim 47, wherein no portion of the second channel is disposed within the first channel when the tip is secured to the probe.

49. The ear probe system of claim 46, wherein the protrusion is angled.

50. The ear probe of claim 49, wherein the protrusion is a ring encircling the pedestal portion.

51. The ear probe system of claim 46, wherein the tip and the probe include a visual guide feature to provide proper radial alignment of the tip to the probe.

52. The ear probe system of claim 51, wherein the visual guide comprises a projection extending from the probe and a corresponding cutout disposed on the tip.

53. The ear probe system of claim 46, wherein the second channel is sized to prevent the first channel from contacting debris in an ear of a subject when the tip is secured to the probe and is positioned in the subject's ear.

54. The ear probe system of claim 46, wherein the signal transducing device is a microphone.

55. The ear probe system of claim 46, wherein the signal transducing device is a speaker.

56. The ear probe system of claim 46, further comprising a light source to aid in a visual inspection of the subject's ear.

57. The ear probe system of claim 56, wherein the light source is a light emitting diode.

58. An ear probe system comprising:
a probe defining a first probe channel and a second probe channel and including a first alignment feature; and
a tip defining a first tip channel and a second tip channel and including a second alignment feature, the first alignment feature of the probe mating with the second alignment feature of the tip to ensure alignment of the first probe channel with the first tip channel when the tip is secured to the probe.

59. An ear probe system comprising:
a probe defining a first probe channel and a second probe channel;
a tip defining a first tip channel and a second tip channel, and
means for automatic alignment of the first probe channel to the first tip channel when the tip is positioned on the probe.

60. An ear probe system comprising:
a probe comprising:
a base portion housing at least one signal transducing device; and.
a cap portion extending from the base portion, the cap portion defining a first probe channel and a second probe channel and including (i) a protrusion and (ii) a tip alignment feature, the tip alignment feature disposed between the first and second probe channels; and
a tip comprising an exterior surface and an interior surface, the interior surface defining a first tip channel, a second tip channel, and a probe alignment slot,
wherein the tip alignment feature has a greater length along a longitudinal direction of the ear probe system than the probe alignment slot so as to stretch the tip over the protrusion during attachment of the tip to the cap portion of the probe.

61. A method of attaching a tip to a probe, the method comprising the steps of:
(a) providing a probe comprising:
a base portion housing at least one signal transducing device; and
a cap portion extending from the base portion, the cap portion defining a first probe channel and a second probe channel and including (i) a protrusion and (ii) a tip alignment feature, the tip alignment feature disposed between the first and second probe channels;
(b) positioning a tip comprising an exterior surface and an interior surface, the interior surface defining a first tip channel, a second tip channel, and a probe alignment slot, the probe alignment slot having a smaller length along a longitudinal direction of the ear probe system than the tip alignment feature of the probe; and
(c) stretching the tip over the tip alignment feature of the probe such that a portion of the tip is secured by the protrusion.
